(19) United States
(12) Patent Application Publication
(10) Pub. No.: US 2015/0309295 A1
(43) Pub. Date: Oct. 29, 2015

(54) MINIATURIZED IMAGING DEVICES, SYSTEMS AND METHODS

(71) Applicant: INSCOPIX, INC., Palo Alto, CA (US)

(72) Inventors: Eric COCKER, Menlo Park, CA (US); Kunal GHOSH, Mountain View, CA (US)

(73) Assignee: INSCOPIX, INAC., Palo Alto, CA (US)

(21) Appl. No.: 14/440,026

(22) PCT Filed: Nov. 5, 2013
(86) PCT No.: PCT/US13/68547

§ 371 Date: Apr. 30, 2015

Related U.S. Application Data
(60) Provisional application No. 61/722,721, filed on Nov. 5, 2012.

(51) Int. Cl. G02B 21/00; G02B 21/36; G02B 21/06; A61B 5/00

(52) U.S. Cl. CPC G02B 21/0076; A61B 5/0071; A61B 5/4064; G02B 21/362; G02B 21/06

(57) ABSTRACT

The invention provides miniaturized devices, systems and methods for imaging of biological specimens. The devices and system provide accurate alignment and modular mounting of imaging components internally and in relation to the target subject. In some embodiments, the invention provides devices, systems and methods for in vivo fluorescent brain imaging in freely-behaving rodents.

### CROSS-REFERENCE

This application claims priority to U.S. Provisional Patent Application Ser. No. 61/722,721, filed on Nov. 5, 2012, which is entirely incorporated herein by reference.

### BACKGROUND OF INVENTION

High performance imaging devices and systems remain bulky and expensive instruments. These constraints increase with increasing imaging complexity, without the ability to easily incorporate additional functionality. Present devices and systems are especially not well suited for distributed, chronic imaging of live biological specimens.

### SUMMARY OF INVENTION

Recognized herein is the need for small, lightweight, customizable and easily assembled imaging devices and systems.

The invention provides devices, systems and methods for miniaturized imaging of biological specimens. Some embodiments provide devices, systems and methods for miniaturized in vivo fluorescent brain imaging in freely-behaving rodents.

An aspect of the invention relates to an imaging device, comprising: a base plate; and a device body, wherein the device body is configured to be connected and separated with the base plate in a reproducible manner. In some embodiments, an imaging device may be provided, comprising: a base plate configured to be attached to a subject having a target region to be imaged; and a device body having an image sensor configured to image the target region when the device body is connected to the base plate, wherein the device body is configured to be connected to and separated from the base plate in a reproducible manner.

In some embodiments, the base plate can comprise one or more subject attachment mechanism configured to attach the base plate to the subject so that the base plate does not move relative to the target region. Optionally, at least one of the base plate or the device body comprises one or more magnets, such that the device body is configured to be magnetically connected to and separated from the base plate. The one or more magnets may be positioned to cause the device body to snap to a particular alignment with the base plate. The base plate and device body may comprise mating surfaces that can mechanically prevent at least one of rotational movement or axial movement between the base plate and the device body when the device body is connected to the base plate.

In some embodiments, the device body has a volume of 10 cubic centimeters or less. The base plate may have a maximum dimension of 3 cm or less. In some instances the device body weighs less than 2 grams.

The device body may have a housing containing the image sensor and one or more optical elements along an image collection pathway from the target region to the image sensor. In some embodiments, the base plate may have a hole and the device body may have an objective lens configured fit at least partially through the hole to capture light from the target region when the device body is connected to the base plate.

Another aspect of the invention provides an imaging device, comprising: a focusing unit; and an imaging body comprising an illumination pathway and a collection pathway, wherein the focusing unit is restrained relative to the imaging body. In some implementations, an imaging device may comprise: a focusing unit having an image sensor configured to image a target region; and an illumination unit comprising an optical element disposed along an image collection pathway from the target region to the image sensor, wherein the focusing unit and the illumination unit are movable relative to one another in an axial direction, and wherein a degree of the movement between the focusing unit and the illumination unit is restrained by a tamper restraint focus lock.

In some embodiments, the tamper restraint focus lock may prevent the focusing unit from being separated from the illumination unit. The tamper restraint focus lock may also include protrusion on an inner surface of the illumination unit and a protrusion extending radially from a surface of the focusing unit. The protrusion on the inner surface of the illumination unit may be a set screw, and a ring may be provided behind the set screw that restricts the set screw's movement.

The illumination unit may have a housing having an illumination source within the housing, configured to provide illumination to the target region via an illumination pathway. The optical element may be positioned along the illumination pathway. In some implementations, the movement between the focusing unit and the illumination unit may result in a change of length of the image collection pathway. The image collection pathway can have a maximum length of less than or equal to 30 mm. In some embodiments, the focusing unit and the illumination unit may be connected via a threaded interface, whereas turning the focusing unit and the illumination unit about the threaded interface effects the movement in the axial direction between the focusing unit and the illumination unit.

The imaging device may have a volume of 10 cubic centimeters or less. The imaging device may weigh less than 2 grams.

An additional aspect of the invention relates to an imaging device, comprising: one or more objectives; and a device body, wherein the one or more objectives are configured to be connected and separated with the device body in a reproducible manner. Aspects of the invention may include an imaging device, comprising: a device body having a volume of 10 cubic centimeters or less, said device body comprising an image sensor configured to image a target region of a subject; and one or more objective lenses disposed along an image collection pathway from the target region to the image sensor, wherein the one or more objective lenses are configured to be connected and separated with the device body in a reproducible manner.

Additionally, one or more objective mounts may be provided for holding and mounting said one or more objective lenses to the device body in a predetermined orientation with respect to the device body. The one or more objective mounts may include one or more magnets that aid in attachment and alignment of the one or more objective lenses to the device body. The imaging device may be configured to accept a plurality of objective lenses having different field of view or resolution characteristics with aid of the one or more objective mounts.

The device body may have a housing containing an illumination source within the housing, configured to provide illumination to the target region via an illumination pathway. The objective lens may be configured to be positioned less than 5 mm away from the target region and provide a focused image to be captured by the image sensor. In some embodiments, a greatest dimension of the device body may be less than 20 mm. Optionally, the imaging device may weigh less than 2 grams.

Other goals and advantages of the invention will be further appreciated and understood when considered in conjunction with the following description and accompanying drawings. While the following description may contain specific details describing particular embodiments of the invention, this should not be construed as limitations to the scope of the invention but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic of a miniaturized imaging device and system in relation to a subject.

FIG. 2 is a cut-away perspective side view of a miniaturized imaging device.

FIG. 3A is an exploded perspective side view of a magnetic quick-release base plate for microscope attachment.

FIG. 3B is a perspective side view of a miniaturized imaging device with a quick-release base plate.

FIG. 3C shows photographs of structural members in a miniaturized imaging device with a magnetic quick-release base plate for microscope attachment.

FIG. 4A is a side view of a miniaturized imaging device with a tamper-resistant threaded focusing unit.

FIG. 4B is a sectional side view of a tamper-resistant focusing unit.

FIG. 5 is an exploded perspective bottom view and an exploded perspective side view of an objective mounting and alignment arrangement on an illumination unit.

FIG. 6A is a perspective side view and a sectional top perspective view of an illumination unit, illustrating an alignment step during objective mounting and alignment.

FIG. 6B is a cut-away perspective side view and sectional top perspective view of an illumination unit, illustrating an insertion step during objective mounting and alignment.

FIG. 7 is a schematic outlining the process flow in an imaging method in accordance with embodiments of the invention.

FIG. 8 shows a miniaturized imaging device assembled on a test rig.

FIG. 9A is an image of yellow fluorescent protein (YFP)-expressing neurons in a mouse brain slice acquired with a miniaturized imaging device and system in accordance with embodiments of the invention.

FIG. 9B is an image of YFP-expressing neurons in a mouse brain slice.

FIG. 9C is an image of YFP-expressing neurons in a mouse brain slice.

### DETAILED DESCRIPTION OF INVENTION

The invention provides miniaturized devices, systems and methods for imaging of biological specimens. In some embodiments, the invention provides devices, systems and methods for in vivo fluorescent brain imaging in freely-behaving rodents. Various aspects of the invention described herein may be applied to any of the particular applications set forth below or in any other type of imaging setting. The invention may be applied as a standalone method or system, or as part of an integrated imaging system. It shall be understood that different aspects of the invention can be appreciated individually, collectively, or in combination with each other.

Any description of alignment and assembly of optical and/or mechanical components for the purpose of miniaturized fluorescent imaging herein may also be applied to alignment and assembly of components (e.g., reflective or refractive optical surfaces such as lenses, mirrors, prisms or combinations thereof, wave guides or cavities, thermal elements, electric current or voltage sources, electronic circuit components such as capacitors, inductors and diodes, electromagnetic oscillators or antennae, gas discharge devices, radiation sources and radiation filters) used in other imaging techniques known in the art. For example, ultrasonic, microwave, thermal, radioactive, electron and/or other type of imaging devices (also referred to herein as “microscopes”) may equally benefit from features described herein.

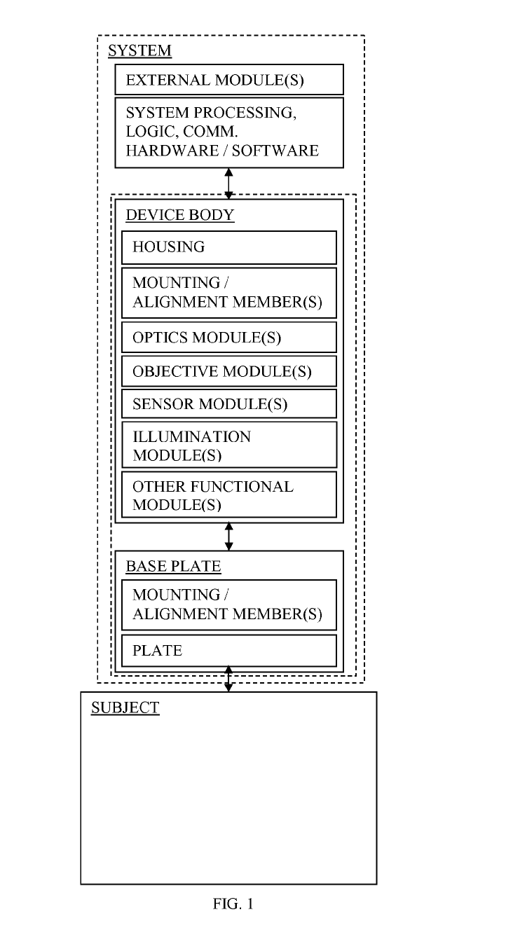

FIG. 1 is a schematic of a miniaturized imaging device and system in relation to a test subject. The imaging device may comprise a base plate mounted to the test subject. In some embodiments, the test subject may be a freely moving animal (e.g., a rodent such as a rat, mice, guinea pig, hamster, gerbil) and the base plate of the device may be mounted to the body of the animal (e.g., the skull, extremities, chest, stomach, spine, joints) in a predetermined location with respect to a target location (e.g., a location in the brain, internal organ, spinal cord, blood vessel, nerve bundle, muscle tissue, bone, skin). The imaging device may or may not be mounted on the base plate. The subject may be substantially mobile. The subject may be capable of ambulating from one location to another. The subject may freely traverse the subject's environment while the base plate and/or the imaging device body is mounted on the subject. In some embodiments, the subject is not anesthetized. The subject may be conscious or awake while the base plate and/or device body is mounted. The subject may be freely moving and/or conscious while the imaging device is mounted on the subject and capturing images from a target area of the subject. The device may be mounted externally on the body of the animal, or internally in the body of the animal (e.g., subcutaneously, operated inside the animal such as near a blood vessel or near an internal organ, on a rib cage or other internal mounting platform). The device may be mounted partially externally and partially internally. For example, some components of the device may be mounted externally for easy access, whereas other components may reside inside the animal.

In some embodiments, test subjects may include, but are not limited to, vertebrates, such as, for example, rodents (e.g., rabbits, rats, mice, guinea pigs, hamsters, gerbils), fish (e.g., zebrafish), birds, frogs, cats, dogs, equines, bovines, porcines, non-human primates (e.g., simians, macaques, marmosets, various types of monkeys baboons, or chimpanzees), or humans, and invertebrates, such as, for example, worms (e.g., waxworms) or insects (e.g., cockroaches, fruit flies).

The imaging device may include a base plate. The base plate may be mounted to the test subject using any suitable means known in the art, including, but not limited to, screws, sutures, adhesives, implants and/or other skin, tissue or bone fastener means. Some fastener means may require that holes be drilled into one or more bones, that the animal be operated on to insert implants, that portions of the skin of the animal be parted or removed and/or other invasive bodily procedures (e.g., using a piercing gun). One or more ties or extensions may be used to wrap around a portion of the subject's body to keep the base plate in place. Any mechanisms, such as those described herein, used to attach the base plate to a subject may be a base plate subject attachment mechanism. The base plate may be configured to be fixedly attached to the subject, so that the base plate does not move with respect to the subject once attached. The base plate may also comprise one or more mounting/alignment members. The base plate may be permanently mounted, removably mounted, mounted for a predetermined period of time before self-detaching, or a combination thereof. Further, the base plate may be designed to be mounted for long periods of time (e.g., one or more years), intermediate periods of time (e.g., one or more months, one or more weeks), short periods of time (e.g., minutes, hours, days), or a combination thereof (e.g., part of the base plate may remain for a long period of time while another part may be removed/come off after a short period of time). A more comfortable or better fitting base plate design may be used for long-term mounting. The base plate may remain on a subject during a course of a study, such as a preclinical or clinical trial.

The imaging device may further comprise a device body. The device body may be mounted to the base plate. The device body may comprise various structural and/or functional members and modules enclosed by a housing. The number of device body components may be predetermined or arbitrary. For example, the housing body may comprise one or more optics modules, one or more objective modules, sensor modules, illumination modules, one or more other functional modules (e.g., additional sensor module, communications module, DNA sequencer module) and one, two, three, four or more mounting/alignment members (e.g., one or more magnetic mounting/alignment members). One or more modules may also be joined in a larger module. Vice versa, a module may comprise several submodules to enhance customization and modularity of the device. The integration of submodules may be permanent or temporary. For example, one or more modules may be swapped out for other modules. If a module ceases functioning, a new module can be brought in to replace the non-functioning module. Thus modules/components of the imaging device may be upgraded without having to replace the entire imaging device. In some embodiments, a power supply may be provided within the device body as a functional module.

The mounting/alignment members may be separately formed and mounted or attached to the housing and/or to one or more of the modules. In some cases, one or more mounting/alignment members may be integrally formed with the housing, or with one or more of the modules. Further, any description of mounting/alignment members located in the device body may also be applied to mounting/alignment members on the base plate. The mounting/alignment members on the base plate may be used for attachment of the base plate to the subject (e.g., support feet, brackets, collars or other features ergonomically shaped to fit the subject) and/or for attachment of the base plate and the device body. Attachment of the device body to the base plate may be accomplished by providing mounting/alignment members on the device body, on the base plate, or on both the device body and the base plate. The mounting/alignment members may include extruded features, as well as receiving indents, grooves, locks, slots, ridges, flanges, snaps, threads, and/or other features. The mounting/alignment members may enable accurate and repeated positioning of modular components within the device body, and of the device body with respect to the base plate. In one example, the device body may be repeatedly attached and/or removed from the base plate with aid of the mounting/alignment members on the device body and/or base plate. The mounting/alignment features may optionally have mating or interlocking features. Portions of the device body may be slid in or out relative to portions of the base plate in order to be mounted to the base plate or removed from the base plate.

The members/modules may be assembled inside a common housing. Alternatively, one or more individual members/modules may have a separate housing. Further, one or more individual members/modules may have a substantially limited housing or no housing. In one example, a mounting/alignment member may be located between separate pieces of housing without being enclosed by any housing. In another example, a communications module or other functional module may be attached to a receiving region on the device body outside of the housing, and may or may not have a separate housing. Thus, the device body may be assembled piece-wise and may vary in size in accordance with customization of the members/modules and the housing components. For example, the device body may include a two-piece housing, wherein the first housing is part of a focusing unit and the second housing is part of an illumination unit. The focusing unit may include one or more optics modules, a sensor module, and one or more mounting/alignment members. The illumination unit may comprise one or more optics modules (at least one of the optics modules comprising a light source), one or more objective modules, and one or more mounting/alignment members. The members/modules may be formed as arbitrary three-dimensional forms, including, for example, elongated shapes with circular, linear, polygonal, or curved cross-sections, substantially flat circular, linear, polygonal, or curved shapes, substantially spherically symmetrical shapes or other forms. The members/modules may or may not have a constant cross section. The members/modules may be formed as solid or hollow shapes. For example, one or more of the mounting/alignment members may be formed as hollow shapes to enable a lighter-weight device.

The housing(s) may provide structural support, alignment and protection of device components inside the housing(s). Individual pieces of housing can be made from materials including, for example, various kinds of plastic, metal, resin, or other organic or inorganic materials. In some embodiments, light-weight housing suitable for chronic experiments may be formed, for example, from any conventional plastic material known in the art, titanium, aluminum or carbon fiber. The housing may be formed from a single material. Alternatively, the housing may be formed from two, three or more distinct materials. Structural features on the housing may be integrally formed. A composite housing may also be formed by permanently or temporarily joining, through any of the attachment techniques described herein, separately formed housing pieces. The housing pieces may or may not be formed from the same material.

The members/modules may be permanently or removably attached to the housing and/or to each other. Permanent attachment may be achieved by using screws, glue or adhesive, welded connections, solder, heat stakes or other permanent fastening approaches known in the art. Modular, removable interconnection may be achieved with suitable mating fasteners, including hooks, latches, grooves, snap fit features (e.g., mechanical or magnetic snap fit features), buttons, twist lock connections or other protrusions and features. In some cases, a compression fit may be achieved between components through suitable mechanical coupling means. Alignment and strong mechanical connection between components may be achieved by forming complementary mating features on the receiving component. For example, grooves on a mating component may be female fittings complementary to one or more male fittings on a receiving component, and protrusions on a mating part may be male fittings meant to twist, slide, retractably click or otherwise connect to female receptacles on the receiving component. The receiving features may be designed to be compatible with and/or take advantage of the internal structure of the device in order to enhance the strength and support of the union. For example, cavities, grooves, slots and other spatial or mechanical features internal to the device body may form receiving regions or mechanical supports for members/modules, wherein improved structural stability, alignment and durability of the device may be achieved.

Interconnection may be made directly between housing(s), module(s), and the base plate, or it may be made through additional connecting parts (i.e., mounting/alignment members or adapters). Connections between members and/or modules may be linear or multidirectional. Mating features or connecting parts facilitating interconnection may themselves be linear or multidirectional. For example, a tee-connector or a four-way connector may be used for planar multidirectional interconnects. Three-dimensional interconnects may also be used.

Members/modules may be required to be attached in a predetermined order. In some embodiments, all members/modules may be attached in a variable order and configuration. In other embodiments, two or more members/modules may need to be attached in a predetermined order. For example, the one or more optics modules and the objective module may need to be positioned to enable a predetermined signal path with respect to the base plate. The predetermined interconnection may require that one or more mounting/alignment members also be placed in a predetermined configuration. Additional members/modules may then be added to the predetermined core device skeleton in any order desired. For example, the remainder of the device body may be assembled to fit a particular form factor in accordance with application constraints.

The customization (i.e., modular assembly, placement of housings, mounting and alignment) and the precise mounting and alignment functionality may enable the small form factor (e.g., less than 10 mm in any given spatial direction) of the miniaturized imaging device described herein. Miniaturization may require that small alignment and mounting tolerances are met for proper operation of the device to achieve high sensitivity imaging. Also, the modular assembly enabled by the precise alignment aids in maintaining the cleanliness of the internal parts of the device. The housing(s) may enable protection of internal components (e.g., from dust, oxygen and/or other contaminants) while maintaining the light weight (e.g., about 0.1-20 grams, 0.5-10 grams, 1-5 grams, 1.5-3 grams, or 2 grams) and small form factor of the device. In some embodiments, the housing may be formed from a material that provides little or no light contamination. The housing may be formed from a black or dark material. The housing may be opaque and may permit little or no light from outside the housing to reach the interior of the housing except through one or more optical element. The housing may have features or materials designed to absorb light. Furthermore, the swappability, and easy alignment and realignment of device components enables the device to only carry functionality on board that is currently in use. Additional components may be added or swapped out as needed without being installed in the device at all times.

The size and/or weight of the device may be decreased in accordance with further miniaturization of the components of the device. For example, miniaturized optical components, power sources, light/signal sources and other components not known in the art today may enable further miniaturization. For example, the device may be made of a size of less than about 20 mm, 15 mm, 12 mm, 11 mm, 10 mm, 9 mm, 8 mm, 7 mm, 6 mm, 5 mm, 4 mm, 3 mm, 2 mm, 1.5 mm, 1 mm, 0.5 mm, 0.25 mm, or 0.1 mm in any given spatial direction (e.g., height, width, length, diagonal, diameter). In some embodiments, the size limits mentioned may be a maximum dimension (e.g., the greatest of the device's height, width, length, diagonal, or diameter). The device may have a volume of less than 15 cubic centimeters, 12 cubic centimeters, 10 cubic centimeters, 8 cubic centimeters, 7 cubic centimeters, 6 cubic centimeters, 5 cubic centimeters, 4 cubic centimeters, 3 cubic centimeters, 2 cubic centimeters, 1 cubic centimeter, 0.5 cubic centimeters, or 0.1 cubic centimeters. The device may have a weight of less than about 10 grams, 7 grams, 5 grams, 4 grams, 3.5 grams, 3 grams, 2.5 grams, 2 grams, 1.5 grams, 1 gram, 0.5 grams, 0.1 grams, 0.05 grams, 0.025 grams, or 0.01 grams, thus enabling mounting onto small test subjects (e.g., fruit flies) or in tight spaces (e.g., inside the test subject). In some examples a dimension of the device may fall between about 1 and 5 mm. The devices herein may also be manufactured on the micro- or nanoscale (e.g., on a chip) using microelectromechanical systems (MEMS) design tools or other manufacturing techniques. Further, the device may sealed, or water tight, to enable mounting while immersed in a liquid (e.g., mounting to a zebrafish swimming freely in water, or mounting internally within the body of the test subject, wherein the device or parts of the device may be surrounded by bodily fluids).

With continued reference to FIG. 1, a miniaturized imaging system may include communication between the device and one or more external modules and/or system components (e.g., system processing, logic and communication hardware/software) not residing on the device. External location of system components may aid in limiting the size and weight of the device. For example, image data acquired at the device may be communicated from the communications module on the device to an external data processing unit.

As defined herein, communication may mean that a signal may travel to/from one component of the invention to another. The components may be directly connected to each other or may be connected through one or more other devices or components. The various coupling components for the devices may include, but are not limited to, the Internet, a wireless network, a conventional wire cable, an optical cable or connection through air, water or any other medium that conducts signals, and any other coupling device or medium. Data and/or signal transfer may be continuous or intermittent. For example, a constant image video stream may be communicated from the device to one or more external system components (e.g., a computer or other processor-based device).

Data may be transferred over a network. The network may include any system for exchanging data or transacting business, such as the Internet, an intranet, an extranet, wide area network (WAN), local area network (LAN), personal area network (PAN), satellite or cellular communication networks, and/or the like. A variety of conventional communications media and protocols may be used for the data links. For example, data links may be an Internet Service Provider (ISP) configured to facilitate communications over a local loop as is typically used in connection with standard modem communication, cable modem, dish networks, ISDN, DSL lines, GSM, G4/LTE, WDMCA, or any wireless communication media. The invention may be implemented using one or more of the following communication protocols: TCP/IP, X.25, SNA, AppleTalk, SCSI, NetBIOS, OSI, GSM, WiFi, Bluetooth or any number of communication protocols. Communications of the imaging device with one or more external system component may occur wirelessly or via a wired connection.

In some embodiments of the invention, an internet protocol (IP)-based network architecture for distributed video microscopy may be implemented. Such a system may include one, two, three, or more miniaturized imaging devices in communication with one or more external system components over an IP network. External system components may or may not be shared by devices. For example, one or more external processor-based devices within the system may be in communication with a plurality of devices over the network. In another example, a device may be in communication with an external system component without other devices in the system also being in communication with the same external system component. Thus, a system may include a plurality of devices, one or more external modules and/or system components residing on the devices and/or one or more external modules and/or system components not residing on the devices. The IP-based network may be an enabling platform for in vivo brain imaging in large numbers of freely behaving rodents, utilizing a plurality of miniaturized imaging devices of the present invention.

The external modules not present on the device may be added or swapped out on the device when desired. Further, the external modules may communicate information/data, analog or digital signals or other signals (e.g., radiation, current) to the device. In some cases, this communication may be wireless (e.g., wireless power transmission). Alternatively, the external modules may have a cabled connection to the device (e.g., a power generator providing a predetermined electromagnetic waveform to the device via a coaxial cable). In some embodiments, responses to the information/data, analog/digital signals or other signals provided to the device may be communicated back to the external modules. For example, a voltage may be measured at the device in response to a current provided from an external module, and the measured voltage may be communicated back to the external module. The external modules may also comprise functionality that interacts with the data stream from the device via the hardware or software system components. In some embodiments, external modules may not be actively in use unless residing within the device body. In some embodiments, external modules may be partially within the device body while having a component that is external to the device body. An external module may or may not be partially or completely insertable into the device body. An external module may include a component that is separable from the device body.

One or more external modules may be provided that provide power to the device and/or other system components. A power supply, whether provided as an external module or internally to the device as a functional module, may be any type of stored energy system or generation device (e.g., capacitor, battery, flow battery, concentration cell, electrolytic cell device, fuel cell, other type of galvanic cell device, generator driven by flywheel and/or prime mover fueled by a gas or liquid and/or compressed air). A power supply may also be a continuously available utility supplied power source. One or more power supplies may be provided within the miniaturized imaging system. Different system components may have individual power supplies. Alternatively, one or more power supplies may be shared between system components. Distribution and location of power supplies may be optimized according to load requirements of individual system components.

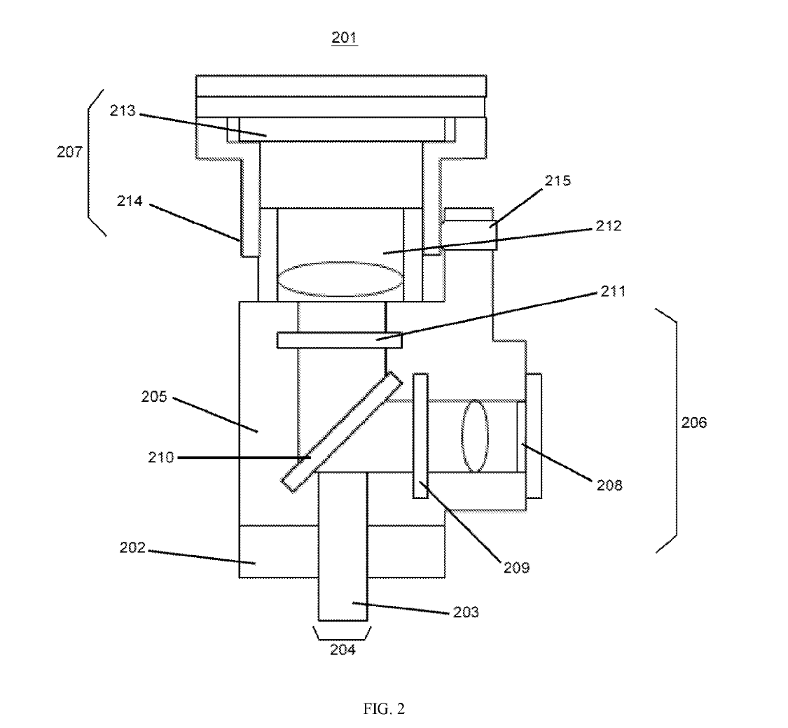

With reference to FIG. 2, an aspect of the invention relates to a miniaturized fluorescence imaging device, such as, for example, a miniaturized imaging device 201 having a width of less than about 15 mm, a depth of less than about 10 mm and a length of less than about 20 mm. Embodiments of the invention may include devices with smaller and/or larger dimensions, such as, for example, devices having dimensions in the range of 0.1-30 mm in any given spatial direction. The device 201 may comprise a base plate 202, configured to be attached to a subject (not shown). An objective 203 may extend through the base plate toward the subject. The objective may be a lens. The objective 203 may have an imaging field of view (FOV) 204. The FOV may be a region of a target that is imaged by the imaging device. Generally, the device 201 may have a housing 205 which may be formed of one, two, three or more separate pieces. For example, separate housings may be provided for an optical unit 206 and a focusing unit 207, wherein each of these housings may further comprise multiple parts.

A light source 208 (e.g., light emitting diode (LED), organic light emitting diode (OLED), laser diode, laser, gas discharge element, or combination or arrays thereof) may reside in the optical unit 206. The light source may be provided within a housing of the imaging device. Any description herein of an LED may apply to any other light source, including those described above. The LED 208 may emit light in a predetermined frequency range. The frequency range of the light from the LED 208 may be selectively narrowed by passing through an excitation filter 209. The resulting excitation wavelength may range, for example, from 460 nm to 500 nm. Alternative configurations of the light source 208, excitation filter 209 and/or additional optical components can permit one or more excitation wavelength ranges to be provided from the optical unit 206. Furthermore, wide or narrow excitation wavelength ranges may be provided (e.g., less than about 100 nm, 75 nm, 50 nm or 25 nm, less than 15 nm, monochromatic light). The electric power to the light source 208 may be varied in accordance with the selected wavelength range(s), desired resolution, FOV and/or other imaging parameters. For example, the power may be about 0.1 mW, 1 mW, 10 mW, 100 mW, 1000 mW or any intermediate value (e.g., 200 mW, 400 mW, 600 mW) or range (e.g., 400-500 mW, 500-600 mW). In some cases, power may be varied or controlled dynamically in accordance with imaging requirements (e.g., power may be adjusted when the imaging parameters of the objective 203 change, such as when one type of objective 203 is swapped by another type of objective 203). The excitation light may then be directed toward a dichroic 210, wherein the light may be reflected in a predetermined direction. As shown in FIG. 2, the excitation light and the dichroic may be arranged such that the excitation light is reflected in a direction parallel to the axis of the objective 203.

The frequency of the excitation light may be in a predetermined range in order to excite fluorescence emission in a target location on the subject (also referred to herein as “sample” or “specimen”). The sample may be made fluorescent through any technique known in the art. For example, a sample may be fluorescent as a result of expression of a fluorescent protein, or the sample may be labeled with fluorescent stains. The excitation light may be passed through the objective lens 203 (e.g., gradient index (GRIN) lens, linear Fresnel lens, collimating lens, or conventional spherical lens) onto the sample, wherein the fluorescence in the sample may give rise to emitted light which may be collected by the same objective 203. The epifluorescent light received by the objective 203 from the direction of the sample may also include excitation light reflected off of the sample. Therefore, the light received by the objective may be passed through the dichroic 210 and further through an emission filter 211 in order to filter out light frequencies not associated with the fluorescence emission from the sample. The emission wavelength may range, for example, from 510 nm to 560 nm.

An achromatic lens 212 and/or one or more other optical elements (e.g., reflective and/or internally reflective elements, refractive and/or internally refractive elements, or prisms) may further focus the emitted light onto an image sensor 213 (e.g., a complementary metal oxide semiconductor (CMOS) sensor). The distance from the achromatic lens 212 to the image sensor 213 may be adjusted through a focusing mechanism 214, which may be configured as a threaded mechanism. The threaded mechanism may comprise additional guiding equipment, such as for example, bearing sets, optical measurement of focusing distance, and other means. The threaded mechanism may for example be configured as a translation stage, wherein a driving motor may rotate a lead screw in order to slide the focusing portion of the device along a shaft utilizing linear motion bearings. Such translation mechanisms may be made very precise, and may be configured to be computer-controlled. Optionally an imaging device housing or body may come in multiple parts. The multiple parts may be threaded and/or configured to engaged in a manner that adjusts one or more dimension of the device housing or body, or an optical path length. In some embodiments, the focusing mechanism 214 may further include a focus lock 215. The focus lock may prevent the housing from coming apart completely, or may provide limits to the degree that the housing dimension and/or optical path length can be varied. The focus lock may provide limits to the degree of focusing that may occur. Such limits may be provided in a single direction or multiple directions (e.g., reduced optical path length, increased optical path length).

Embodiments of the miniaturized imaging device 201 may have an FOV of, for example, 900 μm×700 μm (at middle of focal range), and may provide an average resolution over FOV of about 1.5 μm, wherein the resolution limit of the image sensor 213 may be, for example, on the order of 1.2 μm. In some embodiments, the FOV may be greater than, less than, or equal to about 0.01 mm², 0.02 mm², 0.05 mm², 0.07 mm², 0.1 mm², 0.15 mm², 0.2 mm², 0.3 mm², 0.4 mm², 0.5 mm², 0.7 mm², 1.0 mm², 1.2 mm², 1.5 mm², 2 mm², 2.5 mm², 3 mm², 3.5 mm², 4 mm², 5 mm², 7 mm², or 10 mm². The average resolution may be up to about 250 nm, 300 nm, 500 nm, 700 nm, 1 μm, 1.2 μm, 1.5 μm, 2 μm, 2.5 μm, 3 μm, 3.5 μm, 4 μm, 5 μm, 7 μm, 10 μm, 15 μm, 20 μm, 25 μm, 30 μm, 40 μm, 50 μm, 100 μm, 150 μm, 200 μm, 250 μm, 300 μm, 400 μm, 500 μm, or 700 μm. Any combination of FOV and resolution may be provided. The system imaging resolution can be controlled based on image sensor pixel size (e.g., CMOS sensors with 640×480 pixels=0.3 megapixels, less than 0.3 megapixels, up to 1 megapixels, up to 2 megapixels, up to 3 megapixels, more than 3 megapixels), and/or optical system magnification. In some embodiments, a high degree of resolution may be provided without relying too heavily on optical magnification. For example, the resolutions described may be attained while the optical magnification does not one or more of the following: 1×, 1.5×, 2×, 2.5×, 3×, 4×, 5×, 6×, 7×, 8×, or 10×. In some embodiments, the signal-to-noise (SNR) ratio (i.e., with increasing SNR, controlled for example through signal processing techniques known in the art, corresponding to improved resolution) may be controlled. The SNR may affect effective system imaging resolution (e.g., with deconvolution-based image processing techniques used during post-processing). The overall resolution limit of the device may yield, for example, less than 300 nm, 250 nm, 200 nm, 150 nm, 100 nm, 50 nm, 10 nm or less than about 1 nm precision, depending on imaging technique and image sensor resolution. The overall resolution may be provided at a cellular or subcellular level. In some embodiments, at a subcellular level, details of cells, such as dendrites (e.g., dendritic spines) can be visible.

In some embodiments, the high resolution may be achieved with aid of a short optical path. For example, the distance from a target area to the objective 203 may be less than or equal to 10 mm, 5 mm, 3 mm, 2 mm, 1.5 mm, 1 mm, 0.5 mm, 0.1 mm. Optionally a distance of an optical path from a light source 208 to the objective 203 (e.g., illumination pathway) may be less than or equal to 30 mm, 25 mm, 20 mm, 15 mm, 12 mm, 10 mm, 5 mm, 3 mm, 2 mm, 1.5 mm, 1 mm, 0.5 mm, 0.1 mm. A distance of an optical path from an objective 203 to the image sensor 213 may be less than or equal to 30 mm, 25 mm, 20 mm, 15 mm, 12 mm, 10 mm, 5 mm, 3 mm, 2 mm, 1.5 mm, 1 mm, 0.5 mm, 0.1 mm. An image collection pathway from a target to the image sensor may be less than or equal to 30 mm, 25 mm, 20 mm, 15 mm, 12 mm, 10 mm, 5 mm, 3 mm, 2 mm, 1.5 mm, 1 mm, 0.5 mm, 0.1 mm. In some instances, the maximum length of the image collection pathway, even when the image collection pathway is adjusted, may be less than or equal to 30 mm, 25 mm, 20 mm, 15 mm, 12 mm, 10 mm, 5 mm, 3 mm, 2 mm, 1.5 mm, 1 mm, 0.5 mm, 0.1 mm.

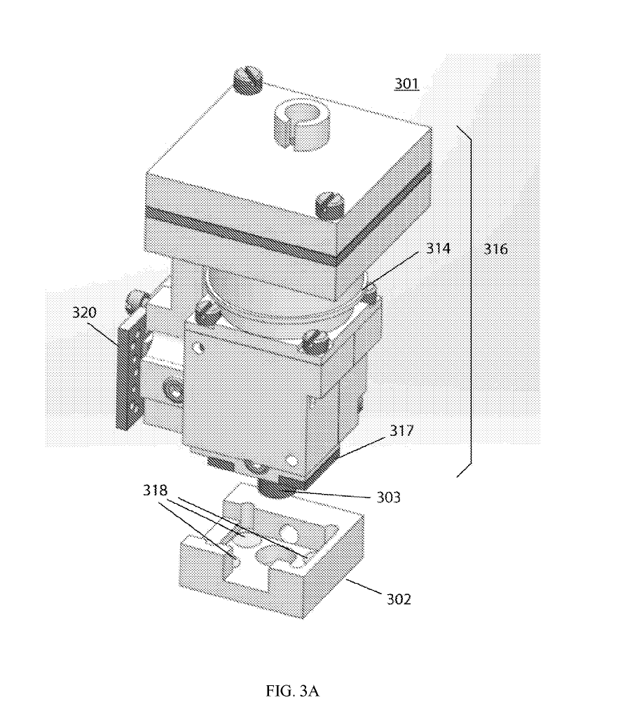

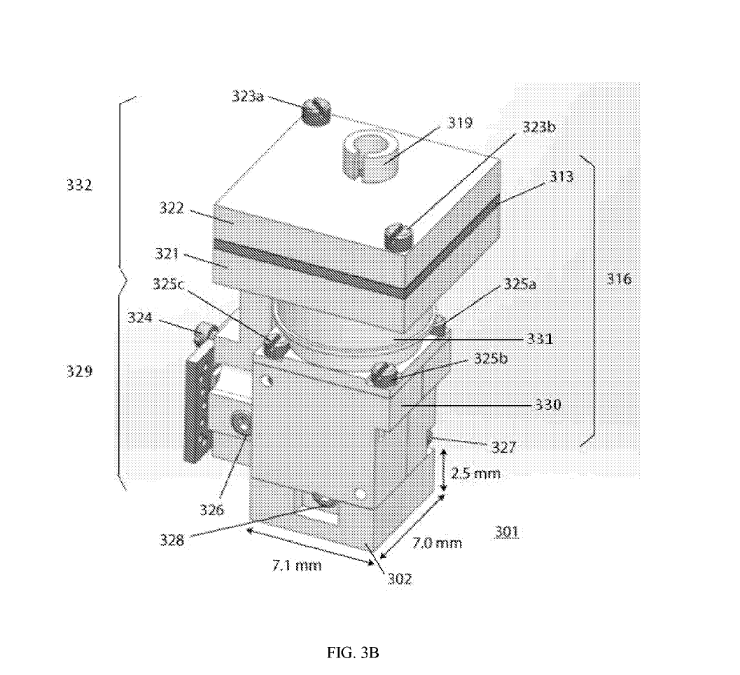

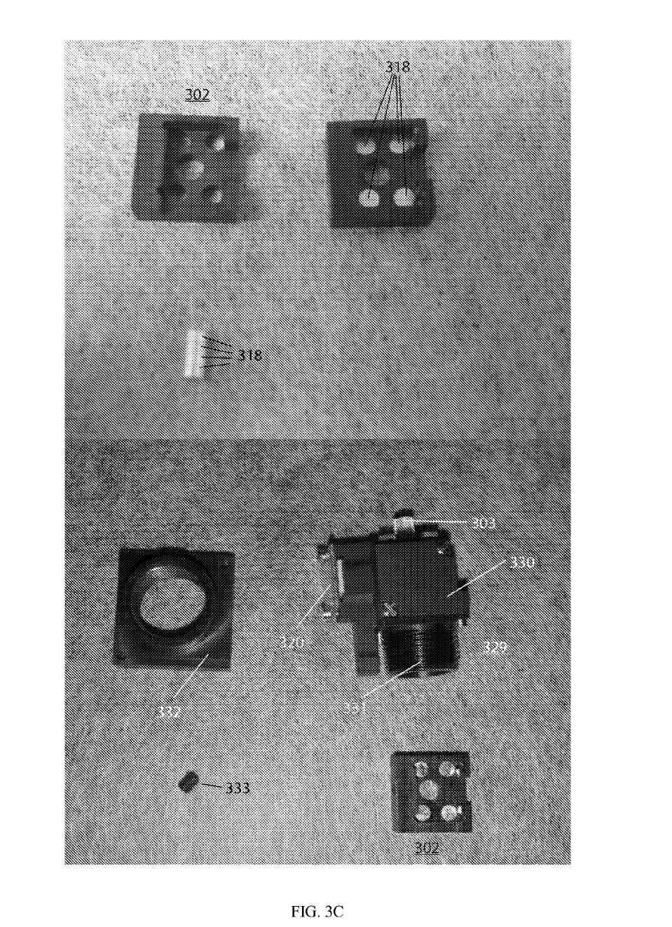

FIG. 3A is an exploded perspective side view of an embodiment of a miniature microscope 301 with a magnetic quick-release base plate 302 for microscope attachment. An objective 303 may be located on a microscope body 316 and may be configured to protrude from the body into an opening provided on the base plate 302. The base plate may be outfitted with one, two, three, four or a larger set of magnets 318. The magnets may be of a flat shape with low thickness, including, for example, circular, square, rectangular and other magnetic plates. The magnets may also have varying thicknesses in the third dimension (e.g., spheres, cubes or cylindrical shapes). Complementary magnet receivers 317 may be provided on the body 316. For example, a set of steel plates 317 may be used to magnetically attach to the magnets 318 on the base plate 302. The magnet receivers may have a cross section that is larger or smaller than that of the magnets 318. The magnet receivers may also exactly match the cross sections and/or spatial form of the magnets 318. For example, cylindrical magnets 318 may attach to half-tubular receivers to provide not only releasable magnetic attachment but also a means of alignment of the base plate 302 with the body 316. In some instances, the body may be attached to the base plate in limited numbers of configurations based on the alignment of the magnets. The body may automatically snap to the appropriate alignment with the base plate in accordance with placement of the magnets.

In some cases, a reverse configuration of the magnets and the magnet receivers may be employed, i.e., the magnets 318 may be provided on the on the body 316 and the magnet receivers 317 may be provided on the base plate 302. In other cases, both the body 316 and the base plate 302 may be outfitted with sets of magnets of opposite polarity. In any of the configurations herein, any number of magnets and/or magnet receivers can be used, such as, for example, one, two, three, four, five, six, ten, dozen, two dozen or more each of magnets and/or magnet receivers. The number of magnets and magnet receivers may be selected to aid appropriate positioning of the body 316 with respect to the base plate 302. The magnets and/or magnet receivers may be positioned to cause the body 316 to snap to a particular spot on the base plate 302. For example, a large concentration of magnets and receivers may be used in the area surrounding the objective 303 rather than at the peripheries. In another example, it may be desirable that the magnets (or any other alternative fastener means described herein) connect the microscope body and the base plate in a predetermined location, such as, for example, in a location where mechanical rigidity is desired while leaving sections of the union more flexible to movement. Alternatively, the magnets and magnet receivers may also be positioned to provide an even force across the joined surfaces. An evenly distributed force may be desirable for example in situations when the alignment of the rest of the device depends on all surfaces being aligned within a predetermined tolerance range. The connection between the base plate and the body may occur with or without the aid of magnets.

The magnets and/or magnet receivers may also be distributed across mating surfaces such that individual sets of magnets/magnet receivers engage in mutually blocking configurations (e.g., one set of magnets/magnet receivers may engage in a predetermined direction while another set may engage in a substantially perpendicular direction to the first set, thereby providing improved restriction of movement in both spatial directions). Various blocking arrangements may be used to restrict linear motion and/or rotation of mating parts. In some embodiments, the blocking arrangements may be provided via mechanical shape of the body and the base plate. For example, a base plate may have a shaped indentation. A corresponding shaped protrusion of the body may fit into the shaped indentation of the base plate. Alternatively, the base plate may have a shaped protrusion that may fit into a complementary shaped indentation on the body. Any combination of interlocking shapes may be provided to further provide alignment between the body and base plate. Such shapes may prevent lateral rotation of the body with respect to the base plate. Interlocking shapes may or may not prevent movement between the body and the base plate in an axial direction. In some embodiments, a body may be positioned on a base plate and then slid or twisted to lock the body into place. Such locking may prevent the body from moving relative to base plate in an axial direction. The sliding or twisting may be reversed to permit the body to be removed from the base plate, and permit such axial movement.

In some embodiments, the attachment mechanisms between the body 316 and the base plate 302 may permit quick attachment and/or release between the body and the base plate. In some embodiments, no separate fasteners or components are required to attach the body to the base plate. The device body may be attached to the base plate with aid of the magnets alone. Alternatively, the device body may be attached to the base plate with only the aid of the magnets and/or one or more integral mechanical shape or feature of the base plate and/or body. The attachment mechanisms may be inherent to the body and the base plate morphology or magnetic qualities. The quick attachment and/or release may be performed without requiring extra tools. A user may be able to attach or release the body from the base plate only using the user's hand.

A ventilation grid 320 may be provided on the body 316 adjacent to a light source (not shown). The ventilation grid may be a heat sink. The ventilation grid may be made from a heat conductor material such as, for example, copper in order to ensure adequate heat transfer from the light source to the surrounding air. In some embodiments, the microscope may be outfitted with a fan or other convective mechanism to enhance the heat transfer rate from the light source.

FIG. 3B is a perspective side view of the miniature microscope 301. A connector or jack 319 may be provided on the microscope body 316 to enable wires, cables and/or other communications means to be connected to an image sensor 313. The connector or jack 319 may be a mechanical reinforcement structure for the cable and attachment point for various components, such as heat shrink tubing, that provides additional mechanical reinforcement. The image sensor may reside between protective housing pieces 321, 322. The housing 322 may have an opening to allow for the wires, cables and/or other communications means to be connected to the image sensor 313. A secure fit of the image sensor may be ensured, for example, through mechanical compression of the pieces 321, 322 by one or more screws 323a, 323b fastener in threaded holes (not shown) provided in the housings 321, 322. The holes may be through holes (e.g., in housing 322). Alternatively, the holes may partially extend though the housing and may not be through holes. In one example, the housing 322 may have through holes while the piece 321 may have through holes or only partially extended (e.g., blind) holes. Additional threaded connections may be employed in the assembly of the microscope body 316, including, for example, one or more screws 324 for holding an illumination source in place, screws 325a, 325b, 325c, 327 for attaching a modular component containing a lens (e.g., tube lens, achromat 212) and/or part of a focusing mechanism and/or other part of a collection pathway, screws 326, 328 (e.g., set screws) for enhancing the mounting and alignment of components within the microscope body. The microscope body, base plate and/or members/modules thereof may be assembled using one or more other mechanical, magnetic or adhesive attachment means described herein. These attachment means may be used in addition to, or as a replacement of one or more of the threaded attachment means on the microscope 301. In some cases, no threaded attachment means may be used to assemble the miniature microscope.

In one embodiment, the quick-release base plate 302 may have a width of 7.1 mm, a depth of 7.0 mm and a height of 2.5 mm. In other embodiments, the dimensions of the base plate may be in the range of 4-10 mm width (e.g., 4 mm, 6 mm, 8 mm, 10 mm width), 4-10 mm depth (e.g., 4 mm, 6 mm, 8 mm, 10 mm depth) and 1-5 mm height (e.g., 1 mm, 3 mm, 5 mm height). In accordance with further miniaturization of the device, the base plate may be made of a size of less than 1 mm in any direction (e.g., less than 0.5 mm, 0.25 mm or 0.1 mm width and/or depth, and less than 0.05 mm, 0.025 mm or 0.01 mm height). In some embodiments a maximum dimension (e.g., greatest of width, depth, or height) of the base plate may be less than or equal to 5 cm, 4 cm, 3.5 cm, 3 cm, 2.5 cm, 2 cm, 1.5 cm, 1.2 cm, 1 cm, 9 mm, 8 mm, 7 mm, 6 mm, 5 mm, 4 mm, 3 mm, 2 mm, or 1 mm. The base plate may weigh 5 grams or less, 4 grams or less, 3 grams or less, 2 grams or less, 1.5 grams or less, 1 gram or less, 0.5 grams or less, 0.3 grams or less, or 0.1 grams or less.

A quick-release base plate 302, such as the magnetic quick-release base plates, may be particularly advantageous in enabling chronic experiments. The magnetic base plate 302 may provide precise, repeatable mounting of the microscope body 316 to a test subject (e.g., the subject's head) for chronic experiments without requiring the use of anesthesia to immobilize the subject. The array of magnets 318 may be located in the base plate in conjunction with the matching set of magnets or ferrous material 317, and the magnetic attachment may provide sufficient normal force to prevent separation of the microscope body 316 from the base plate 302 during an imaging experiment. Side walls on the base plate 302 may restrict lateral linear motion and any rotation of the microscope body so that only force directly opposing the normal force provided by the magnets may separate the microscope body from the base plate (e.g., directly up or perpendicular from the surface to which the base plate 302 is mounted). Additionally, fit adjustment features 328 (e.g., set screws, elastomeric components or retaining springs) may ensure a snug fit between the microscope body and the side walls of the base plate.

The quick-release configuration enables easy removal of the body 316 from the base plate 302. For example, the body may be simply pulled off from the base plate, and then instantaneously re-attached using the automatic mounting and alignment enabled by the quick-release mechanism. In some embodiments, re-attachment may require manual adjustment, while removal may involve simply pulling the microscope body off the base plate. In alternate configurations, the quick-release mechanism may require that a button, spring or other mechanical release feature be pushed or activated in order to release the body from the base plate. In yet other configurations, the microscope body may automatically release itself from the base plate (e.g., using remote control of electromagnets to control the magnetic force, using degradable mechanical linkages that break off after being subjected to a predetermined amount of mechanical stress exerted during movement of the test subject).

The quick-release mechanism may also include multi-step/staged release or multi-step/staged attachment. The microscope body may be removed from the base plate in several steps including, but not limited to, pressing a release feature, followed by twisting or pulling the body 316 off the base plate 302, releasing multiple attachment means (e.g., pressing multiple release buttons), removing a latch, pin or other fastener prior to pulling off the body, etc. Analogously, attachment may also be performed as a sequence of steps. The release and/or attachment mechanism may also be staged. In one example, the microscope body may be partially released from its position before eventually disconnecting either automatically or through mechanical means. For example, electromagnets may be first turned off, causing the microscope body to twist on a hinge while remaining attached to the base plate. The next release stage may lead to permanent disconnection of the microscope body from the base plate, for example through manual release of a connector. In other cases, the quick-release mechanism may involve a procedure wherein the microscope body is pressed toward the base plate before it can be pulled off. For example, the body may need to be pressed toward the base plate to twist, unlock and or otherwise release a fastener (e.g., spring-loaded feature) prior to detachment. Further, in some cases, the body may be removed, and one or more features on the body and/or the base plate may need to be reset (e.g., pulling back a spring-loaded slot or trap feature). The release and/or attachment may also require that additional or replacement parts be supplied. For example, one or more mounting/alignment members may need to be replaced after each removal (e.g., a mechanical member that must break in order for the body to detach).

Benefits of the quick-release configuration include, but are not limited to, enabling the base plate to remain attached on the body of a subject for long term study, easy removal of the microscope body to provide relief to the subject from carrying load while at the same time enabling processing and/or reconfiguring of the microscope body prior to re-attachment, repeated imaging of the same subject (e.g., live being) without the need anesthesia or sacrifice, and enabling imaging during conscious activity.

The microscope body 316 may comprise a body portion 329 and a focusing unit 332. In some embodiments, a microscope body may comprise an illumination unit which may comprise a housing 330 inside which may reside, for example, one or more optics module, an objective module and one or more mounting/alignment members including, for example, the steel plates 317. A flanged mounting/alignment member 331 may be mounted to the housing 330 using threaded attachment means. The mounting/alignment member 331 may have a male tubular threaded portion. The tubular threaded portion of the mounting/alignment member 331 may receive a female threaded portion of a focusing unit 332. The female threaded portion may constitute a portion of the housing of the focusing unit 332. The female threaded portion may have a flange 321.

FIG. 3C shows photographs of structural members of the miniature microscope 301. In the photograph on the left, the base plate 302 is shown without and with four magnets 318. The magnets 318 may be press-fit into openings in the base plate 302. In the photograph on the right, the flanged mounting/alignment member 331 is shown mounted to the housing 330, with the ventilation grid 320 and the objective lens 303 also mounted on the illumination unit 329. The flanged female threaded portion of the focusing unit 332 is shown separately. A set screw 333 may be provided which may provide a snug fit between a housing and a base plate.

The quick-release mounting of the microscope body 316 to the base plate 302 may be achieved through a variety of configurations not limited to magnets. For example, mechanical snap fits, quick-release compression fits, buttons, non-permanent/reusable adhesives, brackets and other fastener means may ensure repeatable attachment of the parts. The microscope body 316 may be attached to the base plate 302 at a single point of attachment, or at multiple points of attachment. For example, the microscope body may be attached to the base plate around the entire perimeter of the interface between the two. In some configurations (e.g., compression fits), o-rings and/or washers may be provided.

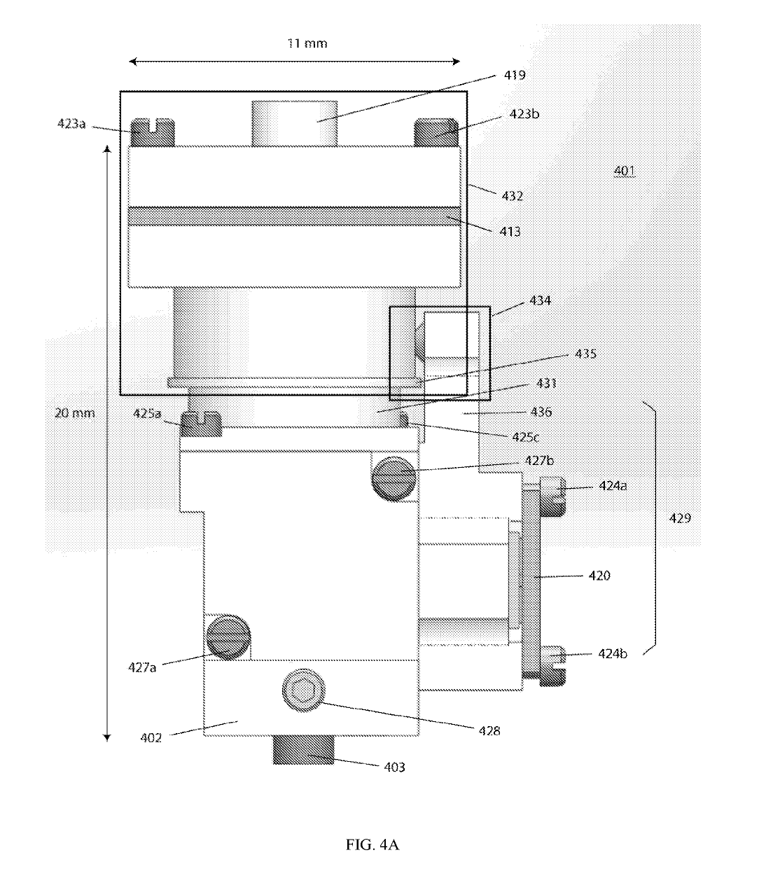

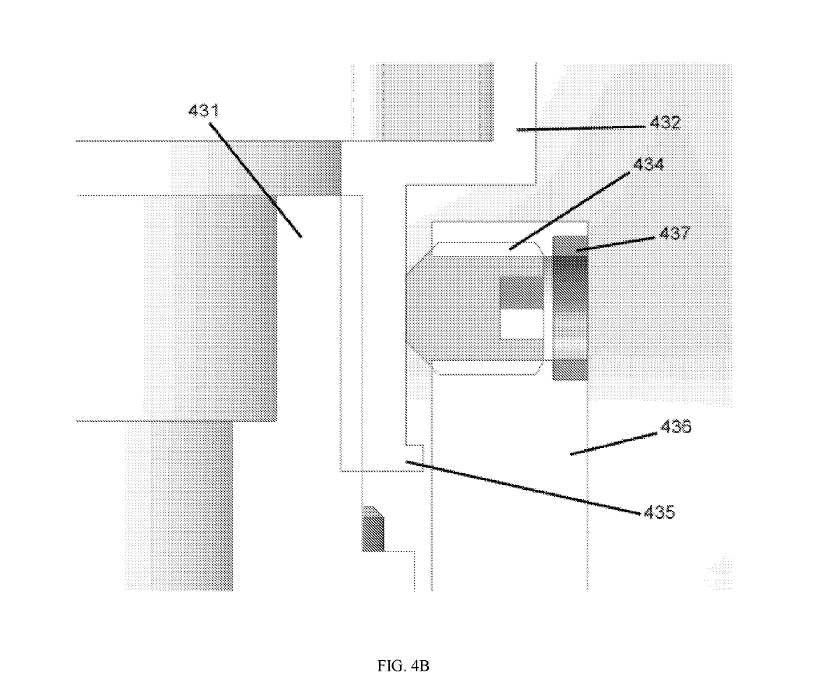

FIG. 4A is a side view of a miniature microscope 401 with a tamper-resistant focusing unit 432 in accordance with another embodiment of the invention. The microscope 401 may have, for example, a width of about 11 mm, and a length of about 20 mm. The microscope may have any dimensions for an imaging system as described elsewhere herein. The microscope may have a focusing unit 432 and an illumination unit 429. The focusing unit and the illumination unit may be movable relative to one another in an axial direction. In some embodiments, they may be movable relative to one another via a threaded connection. The focusing unit may have an image sensor 413 configured to image a target region of a subject. The focusing unit may also have a connector or jack 419 and one or more screws 423a, 423b or other fasteners. Adjustment of the relative positions of the focusing unit and the illumination unit may adjust the optical path length within the microscope. The distance from an objective lens 403 of the illumination unit to the image sensor 419 may be varied.

The tamper-resistant microscope housing assembly may comprise a tamper restraint/focus lock 434 on one or more of the housings of the microscope. The tamper restraint/focus lock may or may not be provided to engage with an illumination unit 429 or other portion of the microscope. The focusing unit 432 may be outfitted with one or more features complementary to the tamper restraint/focus lock 434. For example, the housing of the focusing unit 432 may comprise a flange, ledge, button, pin, bracket and/or other extruded feature 435 for preventing movement of the focusing unit away from the illumination unit past the point of contact with the tamper restraint/focus lock 434. Further, the housing of the focusing unit 432 may comprise a groove, slot, twist lock, and/or other depression or displacement feature for locking the focusing unit in position with respect to the illumination unit at one, two or more predetermined locations. In some embodiments, the focusing unit may be locked in position with respect to the illumination unit at any location accessible through axial movement of the threaded mechanism. Alternatively, the lock-in functionality may also be provided through non-mechanical means, such as, for example, through magnetic attachment. For example, the focusing unit may be made of a magnetically receiving material (e.g., steel or any material with a magnetically receiving coating), and the tamper restraint/focus lock 434 may be a magnet. Conversely, lock-in functionality may be provided such that an extruded or magnetic feature located on the focusing unit engages with a depression or other receiving feature on the illumination unit.

The tamper restraint/focus lock 434 may reside on a mounting arm 436 of the illumination unit 429. The mounting arm may properly position a focus lock set screw.

Control of the relative movement of the focusing unit 432 with respect to the illumination unit 429 may include mechanical, magnetic, electrical forces, chemical (e.g., releasable adhesive) or any combination of these and other techniques and associated features known in the art. The control features may be in contact at one, two or more points along an interface. For example, the tamper restraint/focus lock 434 may not be a button-like extrusion, but may be formed as a collar or bracket around the cylindrical surface of focusing unit. Similarly, in other arrangements of the focusing unit wherein the focusing unit may not be of a substantially tubular shape and wherein a threaded mechanism may not be formed, the restraint/focus lock 434 may be suitably configured to provide similar functionality. For example, if a square tubular arrangement is used, a linear motion assembly may be employed and a square bracket may be used to control the relative motion. As described elsewhere herein, the relative motion may also be computer-controlled via an electric motor. In such configurations, lock-in and positioning may be computer-controlled, and tamper restraint may or may not be provided. Electronic and/or manual control of the focusing mechanism may be calibrated for precise positioning.

The focusing unit 432 may or may not be rotatably suspended to the illumination unit 429. For example, in a threaded configuration, the entire focusing unit 432 may rotate with respect to the illumination unit. Preferably, one or more portions of the focusing unit may remain stationary in the azimuthal direction during axial motion of the coaxial units. For example, a female threaded portion of the focusing unit 432 may be rotatably attached to a flange supporting an image sensor, wherein the sensor may translate in the axial direction during focusing action but may not rotate azimuthally in its plane.

The tamper restraint/focus lock mechanisms described herein may be important for maintaining proper performance and sensitivity in miniaturized imaging devices and systems. Miniaturization may place require low tolerance in optical and sensor assembly combined with stringent cleanliness and sterilization/decontamination standards. As such, opening or otherwise exposing internal parts of the devices or systems to the surroundings may produce deleterious effects, such as, for example, contamination, oxidation or other chemical reaction of optics, sensors and/or other sensitive components, contamination of mechanical members (e.g., dust inside a translation mechanism prohibitive of fine control) and/or other effects. Furthermore, smooth and controlled motion of mechanical joints, threads and other motion control may be important to prevent internal contamination (e.g., dust particles inside the microscope body due to friction on a lead screw during translation and subsequent contamination of grease, silicone, liquids and/or other sensitive internal components). Thus, tamper-proofing and careful control of movement of internal parts may be desirable in these applications.

Without a tamper-resistant threaded focusing mechanism, a user may easily completely unscrew the mating components, exposing the internal optics of the microscope to possible damage. Securing the threaded focusing mechanism may provide a simple way to prevent complete separation of the mating components after the initial assembly process. In some embodiments, the tamper-resistant mechanism may permanently prevent separation after initial assembly. The tamper-resistant mechanism may be designed in a way to accommodate switching of internal components (e.g., modules) without compromising the benefits of isolating internal parts from the surroundings. For example, the tamper-resistant mechanism may remain intact and engaged while one or more internal modules are swapped. In some other embodiments, the tamper-resistant mechanism may be disengaged to allow for internal reconfiguration. Disengagement may be accompanied by preventive measures, such as, for example, closing one or more gates or locks inside the illumination unit 429 and/or the focusing unit 432 in order to prevent contamination. Embodiments of the invention may allow the tamper-resistant mechanism to be disengaged for internal cleaning, repair and/or permanent reconfiguration.

FIG. 4B is a sectional side view of the tamper-resistant focusing unit 432. The focusing component 432, which is threaded internally, may have a small ledge 435 extending radially from its leading edge. After screwing the focusing component onto the externally threaded mating component 431, a small set screw 434, normally used to hold the focusing component at a set location, may be inserted into its mounting arm 436. A small ring 437 may then be secured in a permanent manner (e.g., press-fit) behind the set screw 434, restricting its movement to a range that may only be sufficient for making and releasing contact to the focusing component 432. If the user tries to unscrew the focusing component 432 past a predetermined point, the ledge 435 on the leading edge of the focusing component may come into contact with the protruding set screw 434, thus preventing any further motion. The point at which the ledge comes into contact with the protruding set screw may be matched to the focusing range of the microscope, i.e., the mechanical travel range may be designed to correspond to the range of focusing attainable (also referred to herein as “working distance”). Thus, the ledge-set screw scheme may not only prevent tampering, but may also define the travel range for microscope focusing. The maximum dimension to which a microscope housing may be increased in size (and the maximum length of the optical path) may be determined by the screw set. Tamper-proofing and travel range control may be similarly implemented in non-threaded focusing mechanisms, such as, for example, spring-loaded, hydraulic, linear motion with bearings, telescopic and/or other mechanisms. Optionally, the tamper-resistant mechanism may be disengaged for controlled maintenance by dismantling the ring 437 and/or the set screw 434. Alternatively, the ring and/or the set screw may be permanently affixed to prevent unauthorized parties from dismantling the ring and/or set screw to gain access within the microscope.

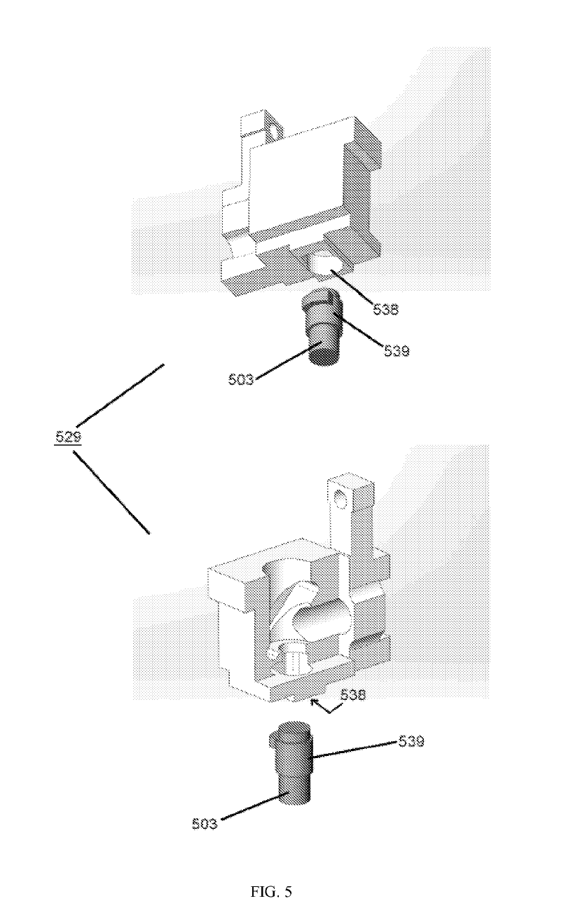

FIG. 5 is an exploded perspective bottom view and an exploded perspective side view of an objective mounting and alignment arrangement on a microscope body 529 in accordance with a further embodiment of the invention. Some imaging tasks may require the ability to switch between low and high resolution objectives on a microscope. The present arrangement may provide a mechanism for easily swapping low and high resolution objectives. The objective swapping mechanism may be implemented without a turret, as motivated by the small form factor of the devices of the present invention. Further, the objective swapping mechanism may rely on precise and repeatable placement of the objectives to ensure that alignment and optical path remain correctly configured when objectives are switched.

A low resolution objective with a larger FOV may be used to observe large structures or to find sparsely populated finer structures. A high resolution objective with a smaller FOV may be used to image the finer structures that may not be resolved with the low resolution objective, but that may have been difficult to find using the smaller FOV of the high resolution objective. In one embodiment of the invention, an average resolution of about 1.5 μm over an FOV of 0.63 mm² may be achieved with an objective with a numerical aperture (NA) of 0.5. A low resolution objective may have a similar or slightly lower resolution due to lower magnification to get a larger FOV. In some embodiments, a high resolution objective may have a diffraction limited resolution of about 0.3 μm, based on an NA of 0.8, over an FOV of about 0.1 mm².

Objectives may be lenses capable of imaging target regions that are close to the lenses. For example, the objective lenses may be placed close to a target region to be imaged on a living subject. The objective lenses may be capable of providing a focused image at one or more of the FOVs or resolutions described herein when less than 15 mm, 10 mm, 8 mm, 7 mm, 6 mm, 5 mm, 4 mm, 3 mm, 2 mm, or 1 mm away from the target region. The focusing of the image at an image sensor may occur with aid of the objective lenses and/or optical set-up of the imaging device.

To easily swap between low and high resolution objectives, the objectives may need similarly conditioned light patterns at their interface with the rest of the microscope's optical system. For example, collimated light may be used. The mechanism may enable multiple objectives to be used. Each objective 503 may have a holder, mount or adapter 539 that may interface with a mounting mechanism to provide simple and precise attachment of the objective to the main microscope body. In some embodiments, the mounting mechanism may include, for example, one, two or a larger set of magnets, a simple swinging clamp, a hand in glove mechanical fit into a cavity, or any other removable attachment means described elsewhere herein.

The objective 503 may be arranged in an objective mount 539 in a variety of configurations, including, but not limited to, press-fit of the objective into a coaxial mount, through adhesives, via pins, heat shrink, latches, flanges on the objective mount secured in mating laser drilled holes, indents or depressions in the objective lens, or any other permanent or temporary attachment means described herein. For example, the objective mount may be tubular and may be formed with a ledge or other mating feature used for mounting and alignment of the objective-mount assembly to the microscope body 529.

Each objective may have different optical characteristics (e.g., FOV, resolution). The objectives may or may not be of the same size (e.g., diameter, length). For example, a thicker coaxial mount may be provided for a smaller diameter objective in order to engage in a constant diameter receiving opening 538. In some embodiments, the diameter of the opening 538 (or other characteristic dimensions in the case of non-circular openings) may be varied in accordance with the diameter of the objective. In other embodiments, a constant outer diameter telescope-like mechanism may be used for mounting the objectives, wherein the telescope can be extended or retracted to make its inner diameter fit a given objective size. The different objectives may have characteristics to allow them to be distinguished from one another. For example, a high resolution objective may have a smaller diameter with respect to a low resolution objective. Alternatively, the objectives may have different geometrical shapes. Fiduciary marks, objective lens color and other physical attributes, including laser or other labeling, can also be used. Such labels may be needed to distinguish objectives with different optical characteristics but same similar physical appearance (e.g., size, color).

Separate objectives may be provided in a variety of configurations with mating receiving features on the microscope body 529. These configurations may allow individual lenses to be swappable and completely separable from the rest of the microscope, i.e., the objective lenses may be stored separately from the microscope body and inserted or removed as desired. In some embodiments, compound (combination) objectives may be provided (e.g., a compound may have multiple lenses and/or other optical elements that may function as a single unit). Compound objectives may be mounted in a similar fashion as separate objectives, wherein the alignment of the compound objective and mount with respect to the receiving opening 538 may be used to select between subobjectives. In some cases, subobjectives may be rotatably arranged within the compound objective mount.

Embodiments of the invention may advantageously provide magnetic or other quick-release mechanisms for alignment and swapping to enable substantial automation of mounting and alignment, and modularity and customization.

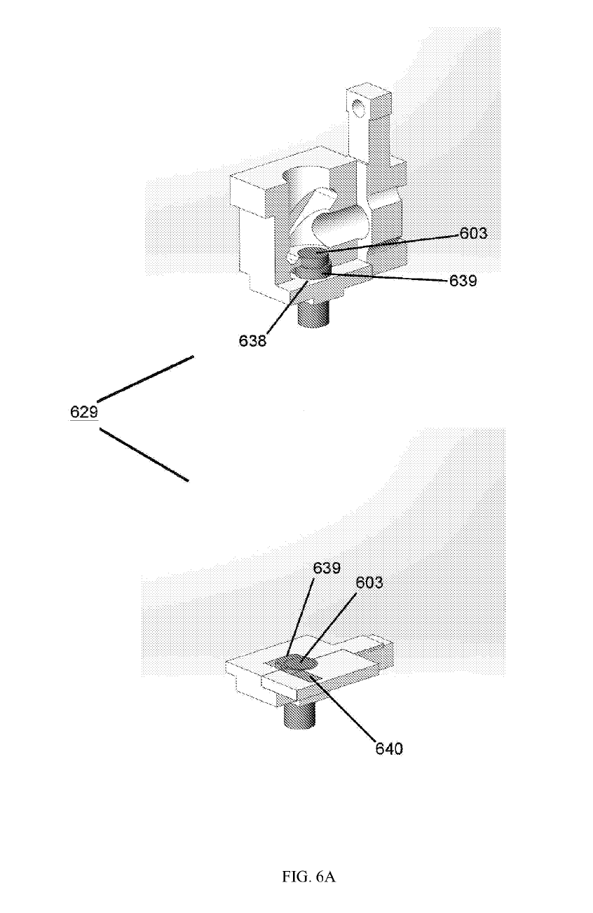

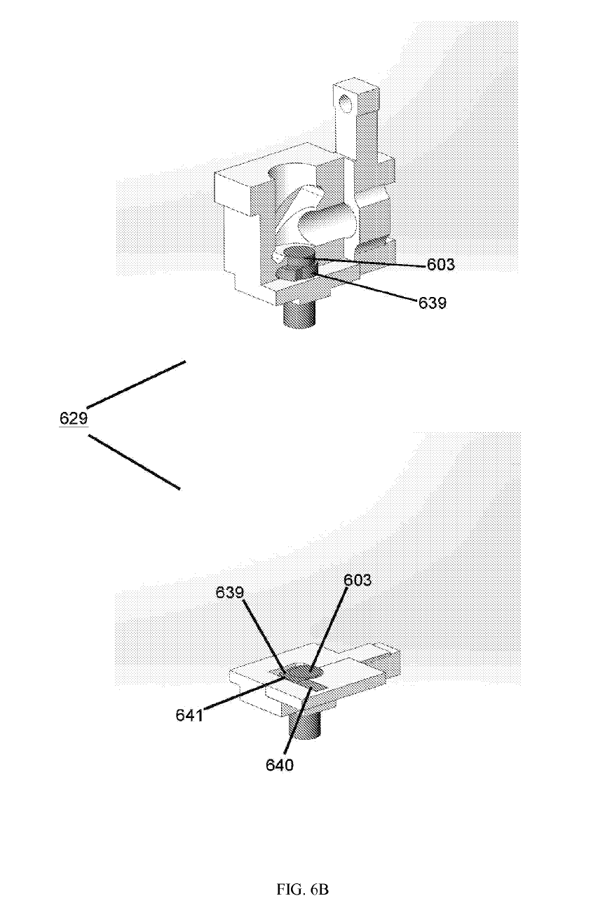

FIG. 6A is a perspective side view and a sectional top perspective view of an illumination unit 629, illustrating an alignment step during objective mounting and alignment. An objective 603 mounted in an objective mount 639 may be aligned in a predetermined orientation prior to insertion into an opening 638 (e.g., a through hole) in the illumination unit. The opening may be shaped to only allow the objective-mount assembly to be inserted if placed in a predetermined relative orientation. In some embodiments, multiple orientations may be possible. The illumination unit may comprise a magnetic member 640 (shown in the sectional top perspective view) of an arbitrary shape (e.g., a magnetic cylinder) oriented in a predetermined direction with respect to the receiving opening and to the intended orientation of the objective. A magnetic force may be established between the objective mount 639 and the magnetic member 640, such as, for example, between a permanent magnet 640 in proximity of a steel or other magnetically active material 639. Alternatively, a reverse configuration may be used, such that the objective mount is of a magnetic material while the receiving member is of a magnetically active material. The objective mount and the receiving member may also both be magnetic with opposite polarity. The attractive magnetic force may cause the objective mount to snap or lock in a predetermined orientation. Additional magnets may be used to enhance the magnetic confinement, such as, for example, in an arrangement where permanently magnetic material may be used to form portions (or all) of the objective mount. The objective mount may experience an attractive magnetic force toward the receiving member (e.g., magnetically active material such as stainless steel, or magnetic material of opposite polarity as the objective mount) and a repulsive magnetic force away from the additional magnet (e.g., magnetic material of same polarity as the objective mount).

FIG. 6B is a cut-away perspective side view and sectional top perspective view of the illumination unit 629, illustrating an insertion step during objective mounting and alignment. The objective-mount assembly 603, 639 may be inserted into the illumination unit with a predetermined alignment of the mounting member ledge on a seat 641 formed in the illumination unit and with respect to the magnet 640 residing in proximity of the seat 641. An initial configuration may be that of the ledge of the objective located at maximum distance from and parallel to the magnet 640. The seat 641 may allow rotation of the ledge toward the magnet, and thus rotation of the objective-mount assembly in a plane perpendicular to the vertical axis of the objective-mount assembly.

Once inserted, the objective-mount assembly 603, 639 may rotate or snap into final position as a result of the attractive magnetic force between the mount 639 and the magnet 640. In some embodiments, the seat 641 may stop the rotating objective-mount assembly in a predetermined position, thus ensuring repeatable positioning of the objective-mount assembly. The seat may set the location of the objective assembly to a predetermined position along an optical axis (e.g., which may be the objective's axis). A tab of the objective mount may be positioned to prevent translational motion along the axis.

In accordance with another aspect of the invention, a method for mounting and aligning miniaturized imaging devices is provided. The method may include mounting and alignment of a miniature microscope body to a base plate on a test subject, tamper-proofing and controlling the travel range of a focusing mechanism, and switching and aligning multiple objectives. Embodiments of the invention enable mounting and alignment of imaging devices onto moving subjects. Further, embodiments of the invention enable modular mounting and alignment, customization and easy swapping of imaging device components. The present method can be implemented during mounting and alignment of various types of imaging devices and/or in other applications requiring accurate mounting and radiation/signal alignment.

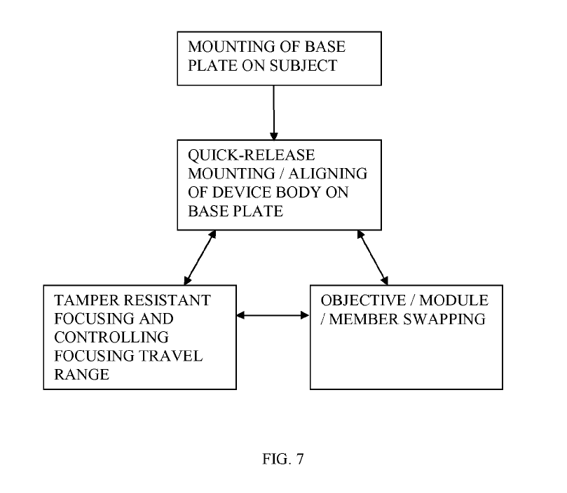

FIG. 7 is a schematic outlining the process flow of the present imaging method. The method may include providing a miniaturized imaging device (miniature microscope) and system. The method may comprise mounting an imaging device body to the base plate, and mounting the base plate on a test subject. In other embodiments, the method may comprise, in a first step, mounting a base plate on a test subject, and, in a second step, mounting and aligning an imaging device body on the base plate. Any of the methods described herein may include unmounting or remounting the imaging device body from the base plate as desired. Additional steps of the method may include focusing the miniaturized imaging device using a tamper resistant focusing mechanism with travel range control capability, i.e., a tamper resistant method for securing and controlling the image focusing mechanism. Additional steps may also include swapping objectives through alignment, insertion and twisting substeps in accordance with FIGS. 6A-6B. Further, additional steps may include swapping modules and/or members on or within the miniaturized imaging device. Each step outlined may comprise one or more substeps. The steps may be repeated and/or performed in a cyclical manner. Feedback loops may exist between the steps. For example, the system may be provided in multiple states (e.g., imaging device mounted on the base plate and imaging device not mounted on the base plate). Focusing may occur while the imaging device is mounted on the base plate. Component swapping may occur while the imaging device is not mounted on the base plate. A user may switch between mounted and unmounted states to perform tasks to reach a desired configuration. Based on images captured, the user may adjust the focus and/or swap components. Further, any of the additional steps may be performed prior or simultaneously with the various steps.

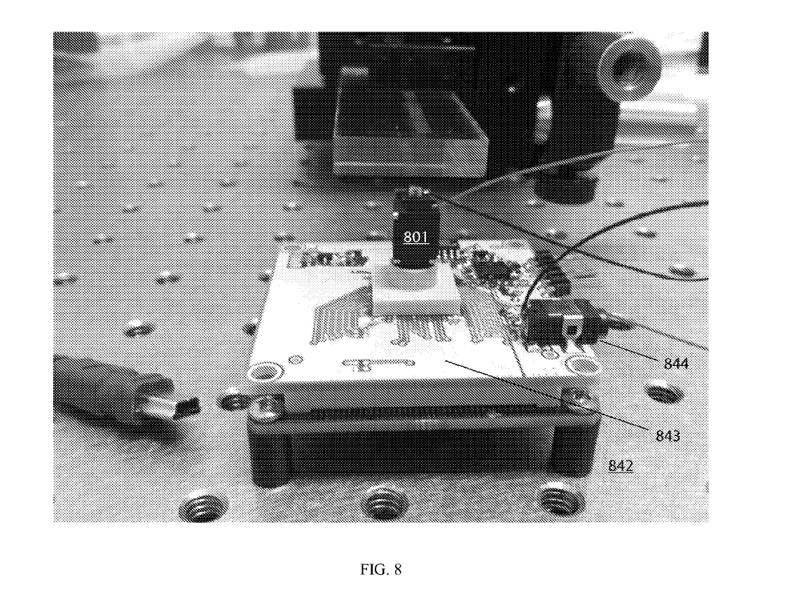

FIG. 8 shows a miniature microscope 801 assembled on a test rig 842. The test rig includes a printed circuit board 843. The microscope may be mounted on a plate, which may provide a function of a housing. The plate may be slightly larger to fit various image sensor packages. The microscope as depicted may be oriented upside down. A power plug (e.g., for an AC adaptor) 844 may optionally be provided. The test rig may be utilized in in vitro scenarios. For example, one or more of the images described herein may be captured using a microscope provided in a test rig as illustrated.

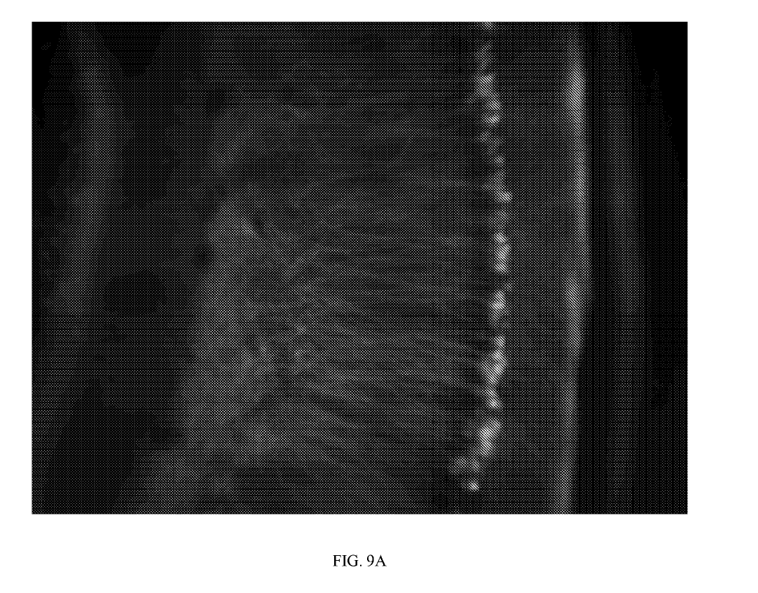

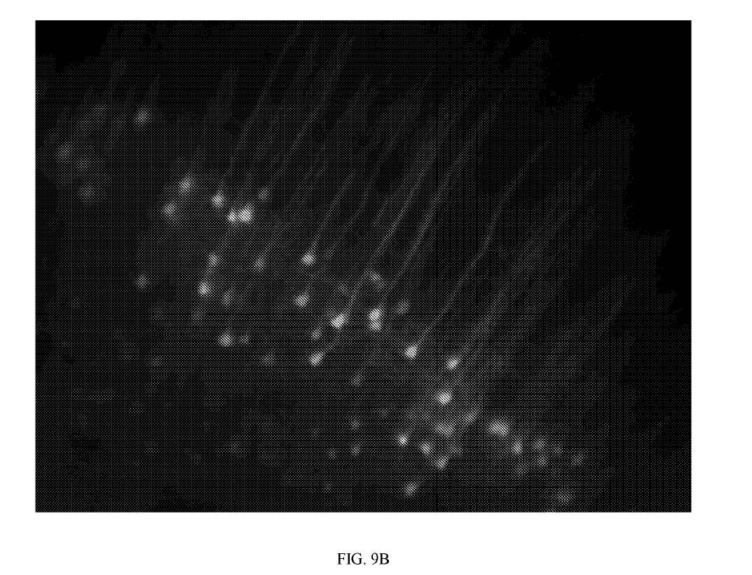

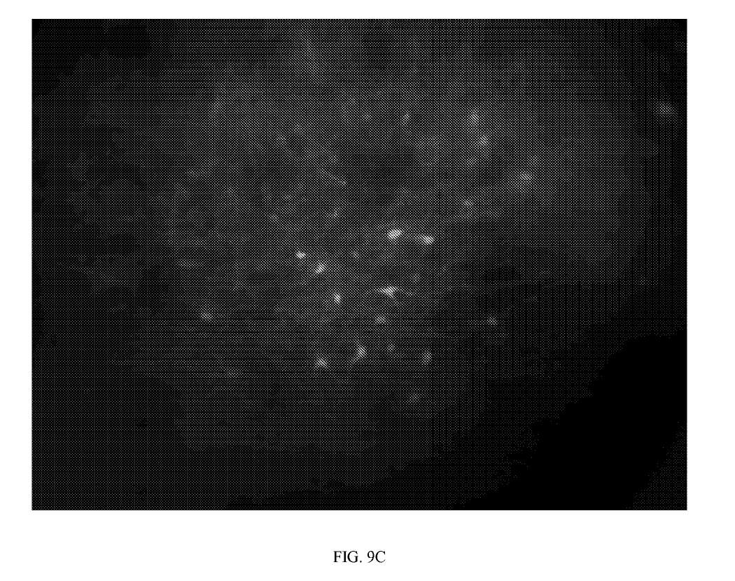

FIGS. 9A-9C show images of yellow fluorescent protein-expressing neurons in a mouse brain slice, acquired with a miniaturized imaging device and system in accordance with embodiments of the invention. The images may be from THY1-YFP expressing neurons. FIG. 9A provides an image of pyramidal neurons from layer CA1 in a hippocampus. FIG. 9B shows neurons from layer 5 of the cortex. FIG. 9C shows an image of layer 2-3 of the cortex. The images were acquired using a miniature microscope with an FOV of about 900 μm×650 μm for all images. The images may be about 1440×1080 pixels (width×height) with an average resolution of less than 2 μm (e.g., about 1.2 μm at the center of the image). The in vitro images in FIGS. 9A-9C may be representative of the types of images that the disclosed miniaturized imaging devices and systems may produce. Further, the images may demonstrate the functionality and imaging performance of the devices (i.e., FOV, resolution, sensitivity).

FIGS. 9A-9C show images of neurons expressing the genetically-encoded fluorescent protein, yellow fluorescent protein (YFP), in a mouse brain slice (THY1-YFP labeling). The present miniaturized devices, systems and methods may equally successfully be applied to imaging of targets labeled with other fluorescent indicators known in the art, including, but not limited to other kinds of fluorescent dyes or genetically-encoded fluorescent proteins, such as, for example, the genetically-encoded fluorescent calcium indicator, GCaMP.

Such images are examples of images that can be captured by the imaging device. Such images may be captured using the imaging device while the imaging device is mounted onto the skull of the mouse, or any other body portion of any other subject. The miniaturized microscope may permit images of such resolution to be advantageously captured while the subject is substantially mobile and free to move about its environment.

The invention may offer significant advantages with respect to existing options for chronic imaging experiments. The imaging system may be easily attached and removed from a subject repeatedly, which are useful for long term studies of living subjects that are free to traverse their environment. Further, the modularity, assembly and operation control provided herein may be needed for successful miniaturization of imaging devices and systems. The systems and methods herein may be advantageously applied to enable ease of assembly and dynamic customization to achieve improved imaging performance.

While preferable embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An imaging device, comprising:
a base plate configured to be attached to a subject having a target region to be imaged; and
a device body having an image sensor configured to image the target region when the device body is connected to the base plate, wherein the device body is configured to be connected to and separated from the base plate in a reproducible manner.

2. The imaging device of claim 1 wherein the base plate comprises one or more subject attachment mechanism configured to attach the base plate to the subject so that the base plate does not move relative to the target region.

3. The imaging device of claim 1 wherein at least one of the base plate or the device body comprises one or more magnets, such that the device body is configured to be magnetically connected to and separated from the base plate.

4. The imaging device of claim 3 wherein the one or more magnets are positioned to cause the device body to snap to a particular alignment with the base plate.

5. The imaging device of claim 1 wherein the base plate and device body comprise mating surfaces that mechanically prevent at least one of rotational movement or axial movement between the base plate and the device body when the device body is connected to the base plate.

6. The imaging device of claim 1 wherein the device body has a volume of 10 cubic centimeters or less.

7. The imaging device of claim 1 wherein the base plate has a maximum dimension of 3 cm or less.

8. The imaging device of claim 1 wherein the device body weighs less than 2 grams.

9. The imaging device of claim 1 wherein the device body has a housing containing the image sensor and one or more optical elements along an image collection pathway from the target region to the image sensor.

10. The imaging device of claim 1 wherein the base plate has a hole and the device body has an objective lens configured fit at least partially through the hole to capture light from the target region when the device body is connected to the base plate.

11. An imaging device, comprising:
a focusing unit having an image sensor configured to image a target region; and
an illumination unit comprising an optical element disposed along an image collection pathway from the target region to the image sensor,
wherein the focusing unit and the illumination unit are movable relative to one another in an axial direction, and wherein a degree of the movement between the focusing unit and the illumination unit is restrained by a tamper restraint focus lock.

12. The imaging device of claim 11 wherein the tamper restraint focus lock prevents the focusing unit from being separated from the illumination unit.

13. The imaging device of claim 11 wherein the tamper restraint focus lock includes protrusion on an inner surface of the illumination unit and a protrusion extending radially from a surface of the focusing unit.

14. The imaging device of claim 13 wherein the protrusion on the inner surface of the illumination unit is a set screw, and the illumination unit further comprises a ring behind the set screw that restricts the set screw's movement.

15. The imaging device of claim 11 wherein illumination unit has a housing having an illumination source within the housing, configured to provide illumination to the target region via an illumination pathway.

16. The imaging device of claim 15 wherein the optical element is positioned along the illumination pathway.

17. The imaging device of claim 11 wherein the movement between the focusing unit and the illumination unit results in a change of length of the image collection pathway.

18. The imaging device of claim 17 wherein the image collection pathway has a maximum length of less than or equal to 30 mm.

19. The imaging device of claim 11 wherein the focusing unit and the illumination unit are connected via a threaded interface, whereas turning the focusing unit and the illumination unit about the threaded interface effects the movement in the axial direction between the focusing unit and the illumination unit.

20. The imaging device of claim 11 wherein the imaging device has a volume of 10 cubic centimeters or less.

21. The imaging device of claim 11 wherein the imaging device weighs less than 2 grams.

22. An imaging device, comprising:
a device body having a volume of 10 cubic centimeters or less, said device body comprising an image sensor configured to image a target region of a subject; and
one or more objective lenses disposed along an image collection pathway from the target region to the image sensor,
wherein the one or more objective lenses are configured to be connected and separated with the device body in a reproducible manner.

23. The imaging device of claim 22, further comprising one or more objective mounts for holding and mounting said one or more objective lenses to the device body in a predetermined orientation with respect to the device body.

24. The imaging device of claim 23, wherein the one or more objective mounts include one or more magnets that aid in attachment and alignment of the one or more objective lenses to the device body.

25. The imaging device of claim 23 wherein the imaging device is configured to accept a plurality of objective lenses having different field of view or resolution characteristics with aid of the one or more objective mounts.

26. The imaging device of claim 22 wherein device body has a housing containing an illumination source within the housing, configured to provide illumination to the target region via an illumination pathway.

27. The imaging device of claim 22 wherein the objective lens is configured to be positioned less than 5 mm away from the target region and provide a focused image to be captured by the image sensor.

28. The imaging device of claim 22 wherein a greatest dimension of the device body is less than 20 mm.

29. The imaging device of claim 22 wherein the imaging device weighs less than 2 grams.
